(19) United States
(12) Patent Application Publication
(10) Pub. No.: US 2026/0325549 A1
(43) Pub. Date: Oct. 22, 2026

(54) OPHTHALMIC COMPOSITIONS

(71) Applicant: OPTIFYE THERAPEUTICS AG, Baar (CH)

(72) Inventors: Minna VAAHTIO, Turku (FI); Lasse LEINO, Masku (FI); Mika KAIMAINEN, Raisio (FI)

(73) Assignee: OPTIFYE THERAPEUTICS AG, Baar (CH)

(21) Appl. No.: 19/106,671

(22) PCT Filed: Sep. 1, 2023
(86) PCT No.: PCT/EP2023/074034

§ 371 Date: Feb. 26, 2025

(30) Foreign Application Priority Data
Sep. 1, 2022 (FI) 20225766

(51) Int. Cl. A61K 31/573; A61K 9/00; A61K 9/06; A61K 9/50; A61K 47/02

(52) U.S. Cl. CPC A61K 31/573; A61K 9/0048; A61K 9/06; A61K 9/501; A61K 9/5084; A61K 9/5089; A61K 47/02

(57) ABSTRACT

This invention relates to a hydrogel silica composition comprising silica particles containing an active pharmaceutical ingredient and a silica sol, such that extended release of the active pharmaceutical ingredient is obtained upon topical ophthalmic topical administration of the composition. The present disclosure further relates to a method for preparing the hydrogel silica composition and its use as a topical ophthalmic composition.

### FIELD

This invention relates to a sustained or controlled release ophthalmic composition for topical delivery. More specifically a sustained or controlled release ophthalmic composition comprising an active pharmaceutical ingredient in a hydrogel silica composition is disclosed herein, which is especially feasible to administer as a topical eye drop, and is suitable for once-a-day administration of the active pharmaceutical ingredient.

### BACKGROUND

Topical ophthalmic drug delivery systems for treatment of ocular inflammation are typically available in the form of dosage forms such as solutions, suspensions, gels and ointments. While these have been found suitable, thus far, for delivery of the required active ingredient to the eye, there are several disadvantages associated with these conventional dosage forms, the most common being the need to administer multiple times a day. The conventional gel and ointment dosage forms are also known to impact vision, or acuity of it, while solution dosage forms are too easily washed off from the surface of the eye. Further, suspension products, if not formulated appropriately, can be gritty, causing injury to the cornea. The pH of ophthalmic solution dosage forms is typically adjusted so as to keep the active ingredient contained therein in solution, but often times this pH is acidic in nature, leading to irritation and redness of the cornea. Moreover preparation of storage stable ophthalmic formulations is also a challenge due to stringent regulatory requirements. These problems grasp one or more conventional ophthalmic dosage forms, creating a barrier in optimum delivery and patient compliance.

Drugs, or active ingredients, that are typically used for treatment of ocular inflammation, such as those arising from disease conditions of the conjunctiva, cornea and anterior segment of the eye, like anterior uveitis, iritis, cyclitis, allergic and vernal conjunctivitis, herpes zoster keratitis, superficial punctate keratitis and non-specific superficial keratitis; corneal injury from chemical, radiation or thermal burns or following penetration by foreign bodies; post-operative use to reduce inflammatory reactions; graft reaction, and the like, include corticosteroids such as dexamethasone and its pharmaceutically acceptable salts, prostaglandins such as bimatoprost, latanoprost and others. Most of these products are commercially available as immediate release dosage forms that require administration several times a day, and may be formulated as solutions or suspensions. Maxidex®, a 0.1% w/v suspension containing dexamethasone is indicated for treatment of steroid responsive inflammatory conditions of the conjunctiva, cornea and anterior segment of the eye, such as, anterior uveitis, iritis, cyclitis, allergic and vernal conjunctivitis, herpes zoster keratitis, superficial punctate keratitis and non-specific superficial keratitis. It is also indicated for the treatment of corneal injury from chemical, radiation or thermal burns or following penetration by foreign bodies, and for post-operative use to reduce inflammatory reactions and suppress graft reaction. The frequency of instillation of these drops and the duration of treatment varies depending upon the severity of the underlying condition and the response to treatment. Severe inflammations require one to two drops of Maxidex® instilled into the eye every thirty to sixty minutes until a satisfactory response occurs. When a favourable response has been observed, the dosage of Maxidex® is reduced towards one drop every four hours. As is obvious, this requires dosing multiple times through the day and can be an issue, in that patient may forget to instil the drops, apart from the sheer inconvenience of instilling multiple times.

Attempts have been made to develop sustained or controlled release dosage forms of dexamethasone, such as a resorbable PEG-hydrogel in the form of an intracanalicular insert to deliver dexamethasone onto the surface of the eye (approved by the US FDA as DEXTENZA®). However, this requires surgical implantation of the insert and cannot be self-administered topically by the patient, or at home. As such, sustained or controlled release ophthalmic compositions which remain stable for long periods during storage are not easy to formulate because of the various complexities involved, such as providing the desired therapeutic efficacy with once daily dosing without impacting safety, ideal pH and viscosity of the composition such that it stays on the surface of the eye for a long period, does not get washed off and yet does not irritate the eye, and a composition that does not impact vision upon administration. There is therefore a need for a stable topical ophthalmic dosage form that overcomes these problems and can be administered once a day.

PCT publication WO2014207304, titled “Silica Hydrogel Composite”, discloses a composition comprising active ingredient encapsulated in silica particles, and suspended in silica sol, which composition is in the form of an injectable, flowable or extrudable composition. The composition provides sustained release of the active ingredient without significant burst, but the publication does not disclose any ophthalmic compositions suitable for administration as an eye drop in the subconjunctival sac. The disclosure of the publication relates to parenteral or surgical implantation products. Nothing in the specification teaches, suggests or motivates formulation of ophthalmic compositions.

PCT publication, WO2017068245, titled “Hydrogel composite depot formulation”, relies on WO2014207304 for the composition of a depot formulation, and focuses on controlled release of an active ingredient from an implant, as compared to conventional depot and microsphere formulations for parenteral administration. The compositions described herein are suitable for once a week to once a year administration. However, nothing in the specification teaches formulation of stable ophthalmic compositions that can be topically delivered. There are many differences between ophthalmic and other parenteral products, such as lipophilic nature of the corneal tissue causes a low affinity environment for the hydrophilic active pharmaceutical ingredients and excipients, faster clearance time from eye reduces the retention time of active pharmaceutical ingredients in the eye thereby causing low bioavailability, and specific viscosity, tonicity and pH requirements for ophthalmic compositions significantly limit the options available to physiochemically stabilize the active pharmaceutical ingredients in such compositions. Because of such differences the teachings of making such parenteral compositions cannot be obviously applied to make stable topical ophthalmic compositions. Ophthalmic topical administration requires several other parameters to be met to be able to be successfully administered to the eye, and to meet sustained or controlled release of the active ingredient contained therein. Ocular implants administered by injection, such as those described in the aforementioned PCT publications, are very different from ophthalmic compositions administered topically as drops that can provide sustained release of the drug contained therein. The latter are neither obvious nor derivable from the former. Both, WO2014207304 and WO2017068245 are incorporated herein by reference, insofar as the technology is concerned.

A February 2019 publication by Nawrat et al (available as https://www.pharmaceutical-technology.com/analysis/delsitech-leveraging-silicas-properties-to-improve-drug-delivery/) discusses about leveraging silica's properties to improve drug delivery. It discloses technology that allows for the controlled release of embedded active pharmaceutical ingredients, and which can thermostabilize the product for years. It discusses that the silica matrix technology could enable vaccines to be stored at room temperature or 4° C., allowing lifesaving medicines to stay effective for far longer. This publication does not discuss sustained release ophthalmic topical compositions, and does not in any way teach use of the technology for obtaining topical eye drops.

The prior art is also replete with examples of ophthalmic compositions of anti-inflammatory agents such as dexamethasone, wherein attempts have been made to sustain or control release of the drug, so as to provide lower number of administrations per day. EP1904108 discloses generally the use of a composition with a permeation enhancer, methylsulfonylmethane, for treatment of disorders, diseases, and other adverse medical conditions, including the adverse ocular conditions disorders often associated with aging. EP3265096 relates to compositions and methods useful for the treatment and/or prevention of conditions of the eye, including dexamethasone as an active ingredient. WO2019126176 relates to a novel mixed transition metal oxide and its use as a catalyst or catalyst precursor such as a hydrocarbon conversion catalyst or catalyst precursor or specifically a hydroprocessing catalyst or catalyst precursor, along with an active ingredient such as dexamethasone. None of these disclosures provide a composition suitable for once daily administration to the eye, in the form of eye drops that can be conveniently administered in the subconjunctival sac.

There is therefore a need for an easy-to-administer stable topical ophthalmic composition, such as an eye drop, that can provide the desired therapeutic efficacy in treating inflammatory ocular conditions, while being administered once-a-day.

### SUMMARY

An objective of the present disclosure is to provide a hydrogel silica composition comprising an active pharmaceutical ingredient for topical ophthalmic administration.

A first aspect relates to a hydrogel silica composition comprising:
a) silica microparticles comprising an active pharmaceutical ingredient, and having a maximum diameter in a range of about 0.5 μm to about 40 μm, and
b) a silica sol comprising solid nanoparticles of <50 nm;
wherein,
i) said silica sol has a solid content of <1% by weight,
ii) said hydrogel silica composition comprises up to 30% by weight of the composition of said silica microparticles, and
iii) said hydrogel silica composition is for topical administration.

In one embodiment, the hydrogel silica composition is a sustained or controlled release composition, preferably the composition is a sustained release composition.

In one preferred embodiment, the hydrogel silica composition is provided in the form of topical eye drops. In another preferred embodiment, the hydrogel silica composition is provided in a single dose container.

In another aspect, the disclosure provides a silica hydrogel composition for use in the treatment of an eye disorder or eye disease. Preferably, the silica hydrogel composition is for topical ophthalmic administration. More preferably, the hydrogel silica composition is topically administered once-a-day.

In one embodiment, the eye disorder or eye disease is ocular inflammation. In a preferred embodiment, the ocular inflammation is selected from anterior uveitis, iritis, cyclitis, allergic or vernal conjunctivitis, herpes zoster keratitis, superficial punctate keratitis or non-specific superficial keratitis, or post-operative ocular inflammation or inflammatory reactions. In a more preferred embodiment, ocular inflammation is post-operative ocular inflammation or inflammatory reaction.

In another embodiment, the eye disorder or eye disease is corneal injury from chemical, radiation or thermal burns or following penetration by foreign bodies.

Another aspect relates to a method of treatment of an eye disorder or an eye disease in a patient in need thereof, the method comprising topically administering to the patient the said hydrogel silica composition. In one embodiment, the eye disease or eye disorder is ocular inflammation selected from anterior uveitis, iritis, cyclitis, allergic or vernal conjunctivitis, herpes zoster keratitis, superficial punctate keratitis or non-specific superficial keratitis or post-operative ocular inflammation or inflammatory reaction, preferably the ocular inflammation is post-operative ocular inflammation or inflammatory reaction. In a preferred embodiment, the hydrogel silica composition comprises dexamethasone or its pharmaceutically acceptable salt as active ingredient.

A yet another aspect relates to the use of the said silica hydrogel composition for treatment of an eye disorder or an eye disease.

Another objective of the present disclosure is to provide a method for preparing the hydrogel silica composition comprising an active pharmaceutical ingredient.

A further aspect relates to a method for preparing a hydrogel silica composition wherein silica microparticles, comprising an active pharmaceutical ingredient, and having a maximum diameter in a range of about 0.5 μm to about 40 μm, are mixed with a silica sol such that
i) said silica sol has a solid content of ≤1% by weight, and
ii) said hydrogel silica composition comprises up to 30% by weight of the composition of said silica microparticles.

In a preferred embodiment, the method relates to preparing a hydrogel silica composition comprising:
a) silica microparticles containing about ≤15 wt-%, preferably 10 wt-%, more preferably ≤7.5 wt-% of dexamethasone or its pharmaceutically acceptable salt, wherein the silica microparticles have an average diameter D10 in a range of 0.9-10 μm and/or average diameter D50 in a range of 0.5-15 μm and/or average diameter D90 in a range of 5-40 μm, preferably 5-20 μm; and
b) a silica sol comprising solid nanoparticles of <50 nm, wherein the silica sol has a solid content of <1% by weight, and
wherein the hydrogel silica composition comprises up to 30% by weight of the composition of said silica microparticles.

In a preferred embodiment of the method, the silica microparticles are obtained by a process of spray drying the silica with the active pharmaceutical ingredient.

Yet another objective of the present disclosure is to provide a hydrogel silica composition obtainable by the method described above.

A preferred aspect relates to providing a hydrogel silica composition obtainable by mixing:
a) silica microparticles comprising an active pharmaceutical ingredient, and having a maximum diameter in a range of about 0.5 μm to about 40 μm, and
b) a silica sol comprising solid nanoparticles of <50 nm;
wherein
i) said silica sol has a solid content of <1% by weight, and
ii) said hydrogel silica composition comprises up to 30% by weight of the composition of said silica microparticles.

Without wishing to be bound by any theory, it is assumed that with the present hydrogel silica composition, sustained release of active pharmaceutical ingredient is reached by embedding an active pharmaceutical ingredient into silica microparticles, and the said microparticles are then mixed with a silica sol to form a semi-solid hydrogel silica composition. The release of the active pharmaceutical ingredient is mainly dependent on the dissolution rate of silica microparticles in the extraocular fluids.

The hydrogel silica composition provided herein is fully biodegradable and bio-dissolvable in body tissues, such as ocular fluids. The biodegradation is based on surface erosion by body fluids, i.e. ocular fluids, and the biodegradation occurs within 24 hours. The drug release from the hydrogel silica composition is strictly controlled by matrix erosion, and is not dependent on solubility of the drug or active pharmaceutical ingredient contained in the silica microparticles. The hydrogel silica composition provided herein is also designed to control or eliminate the initial burst.

In one embodiment, the hydrogel silica composition comprises an active pharmaceutical ingredient selected from anti-inflammatory agents, especially one or more that are conventionally used in treating ocular inflammations like corticosteroids such as prednisolone, dexamethasone, fluocinolone, fluorometholone, medrysone, rimexolone and their pharmaceutically acceptable salts; non-steroidal anti-inflammatory compounds such as ketorolac, flurbiprofen, bromfenac, diclofenac, nepafenac and their pharmaceutically acceptable salts; immunosuppressants such as cyclosporin or voculosporin, antibiotics such as ofloxacin, lymphocyte function-associated antigen-1 (LFA-1) antagonists such as lifitegrast, recombinant-human nerve growth factor such as cenegermin, or other biological drugs used for ophthalmic diseases or disorders. In a preferred embodiment, the active pharmaceutical ingredient is selected from lifitegrast, nepafenac, ofloxacin, cyclosporin, cenegermin or dexamethasone. In a more preferred embodiment, the active pharmaceutical ingredient is dexamethasone or its pharmaceutically salt. Preferably, the ophthalmic composition is administered topically once-in-a-day in the subconjunctival sac.

In a most preferred embodiment, a hydrogel silica composition is provided, wherein the composition comprises:
a) silica microparticles containing about ≤15 wt-%, preferably 10 wt-%, more preferably ≤7.5 wt-% of dexamethasone or its pharmaceutically acceptable salt, wherein the silica microparticles have an average diameter D10 in a range of 0.9-10 μm and/or average diameter D50 in a range of 0.5-15 μm and/or average diameter D90 in a range of 5-40 μm, preferably 5-20 μm; and
b) a silica sol comprising solid nanoparticles of <50 nm, wherein the silica sol has a solid content of <1% by weight, and
wherein the hydrogel silica composition comprises up to 30% by weight of the composition of said silica microparticles.

### DETAILED DESCRIPTION

The hydrogel silica composition provided herein is suitable for topical ophthalmic administration, wherein the rheological properties of the composition are ideal for providing once-a-day administration and sustained release of an active pharmaceutical ingredient contained therein. Further, the content of silica microparticles comprising the active pharmaceutical ingredient can be surprisingly low in the hydrogel silica composition, yet the composition remains stable upon storage for prolonged periods of time and is suitable for ophthalmic uses, preferably suitable for once-a-day administration.

Hydrogel should be understood in the context of this disclosure to be a homogeneous mixture of at least one solid phase and one liquid phase, i.e., a colloidal dispersion, where solid phase(s), e.g., silica (as such and/or as partly or fully hydrolysed) is the continuous phase, and the liquid(s), (e.g., water, ethanol and residuals of silica precursors) is homogeneously dispersed in the structure. The hydrogel silica compositions provided herein have an elastic modulus (G′, which reflects elastic behavior of the composition when deformed) and a viscous modulus (G″, which reflects the flow of the composition while it is deformed) such that the composition exhibits sol-gel properties. Upon application of shear, the composition flows, such as for example when a drop is administered to the eye, and then upon rest in the eye, it gels and increases contact with the eye surface. The hydrogel should therefore be understood to be a gel, where the liquid phase is water or water-based, containing more than 50 weight-% (wt-%) of water, calculated from the total weight of the hydrogel. The liquid phase can additionally comprise other liquids, e.g., ethanol. Accordingly, the term hydrogel and composition are used interchangeably.

The sol should be understood to be a homogeneous mixture of at least one liquid phase and one solid phase, i.e., a colloidal dispersion, where the liquid phase(s), e.g. water, ethanol and possible residuals of silica precursors, is the continuous phase and the solid phase(s), e.g. colloidal particles of silica and/or as partly or fully hydrolysed silica and/or aggregates of said particles are homogeneously dispersed in the said liquid phase. The sol has clear flow properties and the liquid phase is dominating. A suspension can also be called a sol especially if the solid particles are colloidal, and smaller than 1 μm in diameter. In the context of the present disclosure, however, the term sol refers to a colloidal dispersion wherein the solid nanoparticles are ≤50 nm, and the term suspension refers to a dispersion wherein the solid particles are >50 nm.

The term sol-gel transfer refers to a process where a sol turns to a gel, under various conditions. The most typical example of a sol-gel transfer in the present disclosure is that when silica and other corresponding materials (which are synthesised from liquid phase precursors, typically alkoxides and inorganic precursors such as silicate solutions that form after hydrolysis and first condensation particles) are present in the system it is a sol, but once the particles aggregate and/or grow in size, the sol turns to a gel. This can either happen spontaneously (typically in acidic sols) or by induced changes, such as pH change or salt addition (typically in alkaline sols). The sol-gel derived silica can also be prepared by processing to obtain different morphologies, such as by simultaneous gelling, aging, drying and by spray-drying to obtain microparticles.

Shear-thinning refers to an effect where a fluid's viscosity, i.e. the measure of a fluid's resistance to flow, decreases with an increasing rate of shear stress. Shear-thinning in the context of this disclosure is a rheological property of the hydrogel silica composition. Whenever the shear stress or shear rate of such a composition is altered, the composition will gradually move towards its new equilibrium state. At lower shear rates the shear thinning composition is more viscous than Newtonian fluid, and at higher shear rates it is less viscous.

Non-flowing at rest in the context of this disclosure refers to the typical properties of a gel or compositions comprising a gel, where elastic properties (indicated by G′, elastic/storage modulus) dominate over viscous properties (indicated by G″, viscous/loss modulus). In a preferred embodiment, the silica hydrogel compositions disclosed herein have a storage (elastic) modulus G′ higher than loss (viscous) modulus G″. These can be measured with a rheometer, e.g., with a cone-plate or plate-plate geometry within the linear viscoelastic region under small angle oscillatory shear, i.e., the oscillatory shear is so small that in practice it corresponds to properties of the gel, e.g., a hydrogel, or a hydrogel composition at rest.

Accordingly, in a preferred embodiment, the present disclosure provides a hydrogel silica composition comprising an active pharmaceutical ingredient, preferably dexamethasone or its pharmaceutically acceptable salt, wherein storage (elastic) modulus G′ of the composition is higher than loss (viscous) modulus G″.

Silica microparticles in the context of the present disclosure refers to particles of silica preferably prepared by spray drying. Silica microparticles of the compositions provided herein are ≤about 40 μm, preferably ≤about 20 μm, and more preferably ≤about 10 μm in maximum diameter when measured by laser diffraction methodology, e.g., employing a Sympatec HELOS 2370 laser diffraction instrument (see example 5 below). Silica microparticles have a maximum diameter in a range of about 0.5 μm to about 40 μm. According to one embodiment silica microparticles may have a maximum diameter in a range of about 1 to about 40 μm, preferably 1-30 μm, more preferably 1-20 μm, and even more preferably 1-10 μm. According to another embodiment silica microparticles may have a maximum diameter in a range of about 0.9 to about 40 μm, preferably 0.9-30 μm, more preferably 0.9-20 μm, and even more preferably 0.9-10 μm. In yet another embodiment, the silica microparticles may have an average diameter D50 in a range of 0.5-15 μm and/or average diameter D90 in a range of 5-40 μm, preferably 5-20 μm. In a preferred embodiment, the silica microparticles may have an average diameter D10 in a range of 0.9-10 μm and/or average diameter D50 in a range of 0.5-15 μm and/or average diameter D90 in a range of 5-40 μm, preferably 5-20 μm. It has been surprisingly found out that these microparticle size diameter values provide optimal rheological properties for topical ophthalmic administration as well as sustainable release of the active pharmaceutical ingredient enabling for example once-a-day administration.

Silica preferably refers in the context of the present disclosure to amorphous silica, such as amorphous silica containing water, fully or partly hydrolysed amorphous silica or silica in water-dissolved form, such as silicic acid.

R-values referred to in the application, especially in the examples, are defined by the water-to-alkoxide molar ratio of the compositions. Silica compositions may also be expressed with 2 R-values, e.g., RX-Y, where X indicates an initial molar ratio that is used, and Y indicates a total molar water-to-alkoxide ratio after addition of extra liquid, such as water or other liquid, such as ethanol, or ethanol-water mixture, during some stage of the preparation, in a volume that would correspond to the volume of water needed for providing water-to-alkoxide ratio of Y. For example, in R-value R6-50, 6 is the initial molar ratio that is used and 50 corresponds to the total molar water-to-alkoxide ratio after addition of extra liquid during some stage of the preparation in the same volume that would correspond to the volume of water needed for water-to-alkoxide ratio of 50.

In the context of this disclosure, the term active pharmaceutical ingredient (API) refers to any substance or mixture of substances intended to be used in the manufacture of a drug (medicinal) product and that, when used in the production of a drug product, becomes an active pharmaceutical ingredient of the drug product. APIs that can be used on the hydrogel silica compositions of the present disclosure include drugs that are useful as anti-inflammatory agents, especially those that are conventionally used in treating ocular inflammations like corticosteroids such as prednisolone, dexamethasone, fluocinolone, fluorometholone, medrysone, rimexolone or their pharmaceutically acceptable salts; non-steroidal anti-inflammatory compounds such as ketorolac, flurbiprofen, bromfenac, diclofenac, nepafenac or their pharmaceutically acceptable salts; immunosuppressants such as cyclosporin or voculosporin, antibiotics such as ofloxacin, lymphocyte function-associated antigen-1 (LFA-1) antagonists such as lifitegrast, recombinant-human nerve growth factor such as cenegermin, or other biological drugs used for ophthalmic diseases or disorders. Preferably the active pharmaceutical ingredient is selected from lifitegrast, nepafenac, ofloxacin, cyclosporin, cenegermin or dexamethasone. In the most preferred embodiment, the active pharmaceutical ingredient is dexamethasone or its pharmaceutically salt. In a preferred embodiment, the present disclosure provides a topical ophthalmic composition of dexamethasone in a hydrogel silica composition, wherein the composition is suitable for once-a-day administration. Preferably the composition is stable upon storage at 2-8° C. for long periods of time such as at least one month, at least two months or preferably at least three months.

In the context of this disclosure silica composition refers to the hydrogel composition comprising a particular weight percent (wt-%) of silica microparticles, which are combined with a silica sol, resulting in a non-flowing material at rest. Silica hydrogel composition is thus obtainable by mixing the particular weight percent (wt-%) of silica microparticles with the silica sol, resulting in the desired non-flowing hydrogel composition at rest. Percentages by weight are calculated from total weight of the composition, as explained below.

According to one preferred embodiment the hydrogel silica composition is non-flowing upon administration to an eye.

In the context of this disclosure solid content refers to the proportion of non-volatile material contained in a suspension left after the volatile solvent has vaporized. More particularly, it can refer to the solid content of the silica sol used to obtain the hydrogel composition provided herein, or the solid content of the silica hydrogel composition.

The hydrogel composition comprises a particular weight percent (wt-%) of silica microparticles, wherein the wt-% is calculated from the amount of silica particles and silica sol used to obtain the hydrogel composition. Thus, if for example, 100 g of silica microparticles are mixed with 900 g of silica sol, then the wt-% of silica particles in the hydrogel composition is 10 wt-%. If the silica hydrogel composition is obtained by first preparing a suspension of the silica particles, then the percentage is calculated from the original weight of the silica particles in comparison to final weight (i.e. the weight of the silica particles+the weight of liquid used to make a suspension of the silica particles+the weight of the silica sol) of the silica hydrogel composition.

The silica hydrogel composition provided herein, is illustrated by comparing its main features to different materials, e.g., to the properties of the separate components of the composition, such as gels and microparticles and to other prior art gel and hydrogel systems. Gels as such are often used as drug delivery systems, because they are soft, and they can usually be injected into a target tissue or used topically in the form of a sol or a macromolecular solution before they turn into a gel. However, gels usually have a loose structure, which may result in immediate release of the API contained therein. The microparticles, in turn, can be easily combined with water and other liquids to form administrable topical suspensions, but the microparticles can easily flow out of the eye via tear fluid. The present disclosure provides an ophthalmic composition comprising separate components, in which the release of the API is sustained or controlled by silica hydrogel. API release rate is remarkably decreased compared with conventional topical products, such as typical eye drops without sustained-release properties. The preferable type of the ophthalmic composition is a composite of different silica morphologies, that together in an integrated structure provide unique sustained-release properties, i.e. provide synergy, as compared to the individual silica morphologies, so as to provide a matrix for designed, sustained release of the drug or API contained therein. One of the advantageous features of the compositions provided herein is that the combined compositions are easy to handle, and it is easy to mix the separate components into a homogeneous and easily administrable topical ophthalmic composition.

The typical components of the ophthalmic compositions provided herein are silica-based microparticles and a sol comprising silica nanoparticles. After combination of the components and upon topical application to the eye, an integrated structure is formed that can be defined to be a hydrogel. The gel is a silica-based hydrogel. In the compositions provided herein a typical gel consists of a continuous solid phase with liquid homogeneously dispersed within the solid phase, where the elastic/storage modulus of the material is higher than viscous/loss modulus, indicating that the composition is non-flowing at rest. The composition provided herein is typically a gel both before and after administration to the eye.

One of the important properties of these ophthalmic compositions is that they are easily administrable as eye drops and can be provided as a single-dosing unit (SDU), as illustrated in the examples. In a typical single-dose unit for eye drops, a drop is administered from the unit, and the unit is then discarded. The ophthalmic compositions are administrable and flowing because they have shear-thinning properties. The sol-gel property of the ophthalmic compositions ensures that the composition is thinned and flows easily at the time of administration from the SDU, but forms a gel upon instillation or administration to the eye. This gelling prevents the composition from getting washed away by the ocular fluids, and helps in providing a sustained release of the drug, thereby ensuring once-a-day administration.

The hydrogel silica compositions contain silica microparticles in an amount not more than 30 wt-% of the total combined formulation, combined with a sol having typically a low silica content, i.e. solid content, of less than 1 wt-% of silica.

The silica microparticles may comprise up to 30 wt-%, preferably in a range of 0.1-30 wt-%, more preferably 0.5-20 wt-%, even more preferably 1.5-15 wt-%, and most preferably 3-7.5 wt-% of the active pharmaceutical ingredient, such as dexamethasone or its pharmaceutically acceptable salt. The silica microparticles contain typically ≤15 wt-%, preferably ≤7.5 wt-% of API, such as dexamethasone or its pharmaceutically acceptable salt.

The silica microparticles may have an average diameter D10 in a range of 0.9-10 μm and/or average diameter D50 in a range of 0.5-15 μm and/or average diameter D90 in a range of 5-40 μm, preferably 5-20 μm. D10 indicates the diameter value, where 10% of microparticles have a diameter smaller than then D10 diameter, D50 indicates the diameter value, where 50% of microparticles have a diameter smaller than the D50 diameter, and D90 indicates the diameter value, where 90% of microparticles have a diameter smaller than the D90 diameter. Preferably, the silica microparticles are of a size not more than 20 μm, more preferably not more than 10 μm.

In a preferred embodiment, the disclosure provides a hydrogel silica composition comprising:
a) silica microparticles containing about ≤15 wt-%, preferably 10 wt-%, more preferably ≤7.5 wt-% of dexamethasone or its pharmaceutically acceptable salt, wherein the silica microparticles have an average diameter D10 in a range of 0.9-10 μm and/or average diameter D50 in a range of 0.5-15 μm and/or average diameter D90 in a range of 5-40 μm, preferably 5-20 μm; and
b) a silica sol comprising solid nanoparticles of <50 nm, wherein the silica sol has a solid content of <1% by weight, and
wherein the hydrogel silica composition comprises up to 30% by weight of the composition of said silica microparticles.

The silica microparticles employed for preparing the hydrogel silica composition of the present disclosure are microparticles preferably having a maximum diameter in a range of from about 0.5 μm to about 40 μm, more preferably from about 0.9 to about 30 μm, even more preferably from about 0.9 μm to about 20 μm, and most preferably from about 0.9 μm to about 10 μm. The silica microparticles employed for preparing the silica hydrogel composition may comprise up to 30 wt-%, preferably ≤15 wt-%, more preferably ≤7.5 wt % of active pharmaceutical ingredient, such as dexamethasone or its pharmaceutically accepted salts.

According to one preferable embodiment, the active pharmaceutical ingredient is dexamethasone or its pharmaceutically acceptable salt.

In a preferred embodiment the silica sol has a solid content of ≤1 wt-%, wherein the solid nanoparticles are of a size less than 50 nm.

The hydrogel silica composition provided herein is for topical ophthalmic administration. Typically, the use of the hydrogel silica composition is for an eye drop formulation. According to one embodiment, an ophthalmic formulation comprises or consists of the hydrogel silica composition provided herein.

According to one embodiment, the hydrogel silica composition is for use in treatment of an eye disorder or an eye disease by topical administration.

According to another embodiment, the hydrogel silica composition is for use in the treatment of ocular inflammation, preferably ocular inflammation selected from anterior uveitis, iritis, cyclitis, allergic or vernal conjunctivitis, herpes zoster keratitis, superficial punctate keratitis or non-specific superficial keratitis.

According to one embodiment, the hydrogel silica composition is for use in the treatment of corneal injury from chemical, radiation or thermal burns or following penetration by foreign bodies, or for use in the treatment of post-operative inflammatory reactions.

The hydrogel silica compositions disclosed herein are preferably stable upon storage at 2-8° C. or 25° C./60% relative humidity (RH) for prolonged periods of time. Preferably the composition is stable for a period of at least one month, more preferably at least two months, and even more preferably the composition is stable for at least three months upon storage at 2-8° C. The storage stability of ophthalmic compositions is particularly challenging and is determined based on multiple parameters such as changes in appearance, assay % of active pharmaceutical ingredient, pH, related substance analysis, particle size distribution etc., as compared to the initial value. Storage stability of some of the representative hydrogel silica compositions are demonstrated in the Examples.

In highly preferred embodiments, the silica microparticles are obtained by a process of spray drying the silica microparticles with the API, preferably dexamethasone or its pharmaceutically acceptable salt.

The hydrogel silica composition is preferably obtained by mixing the silica microparticles with the silica sol.

Thus, the present disclosure provides a formulation for topical ophthalmic administration which is silica microparticle-silica hydrogel composition, with a surprisingly low total silica content yet a stability of at least 3 months when stored at 2-8° C. and suitability for once a day administration. These hydrogel silica composition provide sustained-release of the API contained therein, while being easy-to-administer topically, surprisingly as a once-a-day eye drop composition. According to one preferable embodiment the hydrogel silica composition may be administered once-a-day to a patient in need thereof.

### FIGURES

FIG. 1 illustrates the cumulative silica dissolution rates in vitro in sink conditions for microparticle formulation (R3-100) with 4 different dexamethasone loading.

FIG. 2 illustrates the cumulative dexamethasone release rates in vitro in sink conditions for microparticle formulation (R3-100) with 4 different dexamethasone loading.

FIG. 3 illustrates storage modulus for 3 different formulations (Formulations #04D-0.3, #06D-0.25, #06D-0.3) at room temperature (ca. 25° C.).

FIG. 4 illustrates the storage and loss moduli for formulation #06-0.25.

FIG. 5 illustrates dynamic viscosity with a thixotropic behavior of #06D-0.3.

FIG. 6 illustrates average dexamethasone concentrations in tear fluids for formulations #04D-0.3, #06D-0.25, #06D-0.3 and #06D-0.35.

FIG. 7A illustrates average dexamethasone concentrations in tear fluids for formulations #09D-0.3 and #12D-0.3 and Maxidex® eye drop product, with single dose.

FIG. 7B illustrates average dexamethoasone concentrations in tear fluid after single dose administration of formulation #09D-0.3 and multiple doses of Maxidex® eye drop product.

FIG. 8: Cumulative in vitro in sink dissolution of silica hydrogel microparticle formulation #09 of Dexamethasone after 3 months of storage at 2-8° C.

FIG. 9: Cumulative in vitro in sink dissolution of silica hydrogel microparticulate formulation of Ofloxacin.

### EXAMPLES

### Example 1

Preparation of Silica Hydrogel Composites from Silica Microparticles (MP) with Encapsulated Dexamethasone and Silica Sol (SS)

The sol-gel derived silica microparticles (MP) were prepared using TEOS (tetraethyl orthosilicate, also known as tetraethoxysilane, available from Sigma-Aldrich) as a precursor for silica. Several microparticle batches with encapsulated dexamethasone with different formulations were prepared with the same general procedure. The initial ratio of water to TEOS (molar ratio) varied from 3:1 to 5:1, depicted as R3 to R5. The initial pH in every sample was adjusted to pH 2 using 0.1 M HCl. The hydrolysis was let to occur at room temperature (i.e. at about 21° C. to about 23° C.) for 25 minutes, under continuous mixing. A solution of dexamethasone in ethanol was cooled to 0° C. and was added to the sol, which was also cooled down to 0° C. The pH of the mixture of silica sol and dexamethasone in ethanol was adjusted to pH of about 3 to about 4 using 0.1M NaOH. Loading percent of dexamethasone in the final microparticles varied between about 2% to about 15% w/w (calculated in relation to the theoretical silica amount). After hydrolysis the sols were diluted by the addition of ethanol (including dissolved dexamethasone) such that the ratio of water to TEOS equals 100, depicted by R100 (i.e., same volume of ethanol was used as water to obtain R100 from the initial ratio of between 3 and 5). For example, a formulation “R3-100 MP” describes a spray-dried silica microparticle formulation where the initial R of the silica sol is 3 and after the dilution with ethanol the R is 100, meaning that the same volume of ethanol is added as water to obtain R100. Every sol was spray-dried to microparticles immediately after the adjustment of the pH using a Büchi B-290 spray dryer (Spray-Dryer parameters: Inlet temperature: 100° C.; Outlet temperature: 68-74° C.; Aspirator: 35 m³/h; Feed flow: 5.6 ml/min; Atomization air flow: 670-700 l/h). The silica microparticles (MP) encapsulated with dexamethasone were thus obtained.

The silica sols (SS) to be mixed with the spray-dried silica microparticles (MP) with encapsulated dexamethasone were prepared using TEOS as a precursor. R of 400 (corresponding to about 0.9% w/w of silica in the silica sol) was prepared. The initial pH of every sample was adjusted to pH 2 using 0.1 M HCl. The hydrolysis was allowed to occur at room temperature (i.e. at about 2° C. to about 23° C.) for 25 minutes under continuous mixing. The pH was then raised to about 5.8 to about 6.2 by adding 0.1 M NaOH with continuous stirring. After the pH adjustment, the silica sols were immediately mixed with the spray-dried microparticles.

The silica microparticles encapsulated with dexamethasone (MP) were added to the silica sol (SS) in an amount varying between 0.1 g to about 0.5 g silica microparticles (MP) in 1 ml of silica sol (SS). The formed silica microparticle-silica sol suspensions were transferred into syringes (1 ml BD luer-lock). The silica microparticle-silica sol suspension in the syringes were kept in a rotating carousel mixer at room temperature, and they turned into a non-flowing gel (a silica hydrogel composite) within 1-3 days. After the gel formation, the formed silica hydrogel composite was transferred by injection (through a 20G needle) into single dose units (SDU, 0.6 ml, Lameplast). The single dose units were stored in aluminium foil in a refrigerator at 2-8° C.

### Example 2

### Dissolution Measurements In Vitro in Sink Conditions for Dissolution Rate of Silica and Release Rate of Dexamethasone

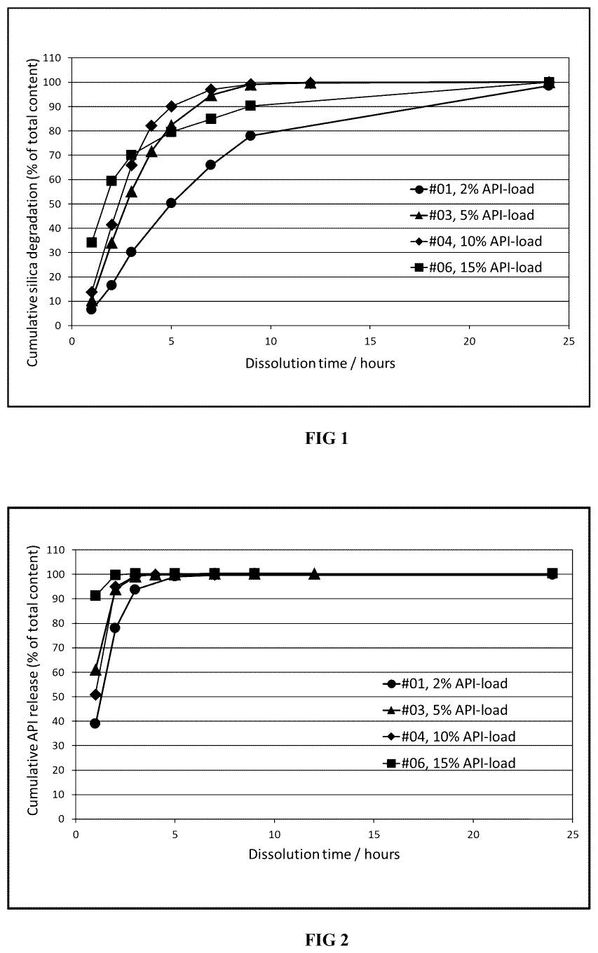

5 microparticle formulations (R3-100 of pH 4.0 with different dexamethasone loading percent as shown in Table 1 and Formulation #09) were selected for in vitro measurements in sink conditions. The dissolution rate of the silica matrix and the release of the dexamethasone were tested by incubating the silica microparticles in a dissolution medium containing 50 mM TRIS buffer (pH 7.4 at 37° C.) placed in a shaking water bath (60 strokes/min). Three replicate samples were collected at each time point for the measurements. The measured dissolution rate of silica and release rate of dexamethasone are illustrated in FIGS. 1 and 2, respectively. FIGS. 8A and 8B provides dissolution rate of silica and release rate of dexamethasone for formulation #09. This is a suitable model for ocular use of the ophthalmic composition provided herein.

TABLE 1
Microparticle formulations for in vitro
measurements in sink conditions

| Microparticle | | | Dexamethasone | |
|---|---|---|---|---|
| formulation | Microparticles | Diluent | load* | pH |
| Formulation #01 | R3-100 | Ethanol | 2% | 4.0 |
| Formulation #03 | R3-100 | Ethanol | 5% | 4.0 |
| Formulation #04 | R3-100 | Ethanol | 10% | 4.0 |
| Formulation #06 | R3-100 | Ethanol | 15% | 4.0 |
| Formulation #09 | R3-200 | Ethanol | 7.5% | 4.0 |

*Loading-% (w/w) in relation to the theoretical mass of silica in microparticles

### Example 3

Rheological Characterization of Hydrogel Composites Containing Silica Sol (SS) and Silica Microparticles (MP) Encapsulated with Dexamethasone

Rheological measurements were conducted with a rheometer (AR 2000 Ex, with a plastic plate measuring head having diameter of 60 mm, TA instruments, Germany) to measure storage (elastic) and loss (viscous) modulus (with oscillatory mode), and dynamic viscosity and thixotropic behaviour (with rotational mode) for the different compositions. The hydrogel composites were injected directly from the single-dosing unit (SDU) onto the measuring plate of the rheometer in order to simulate the properties of the hydrogel composite in real operating situation.

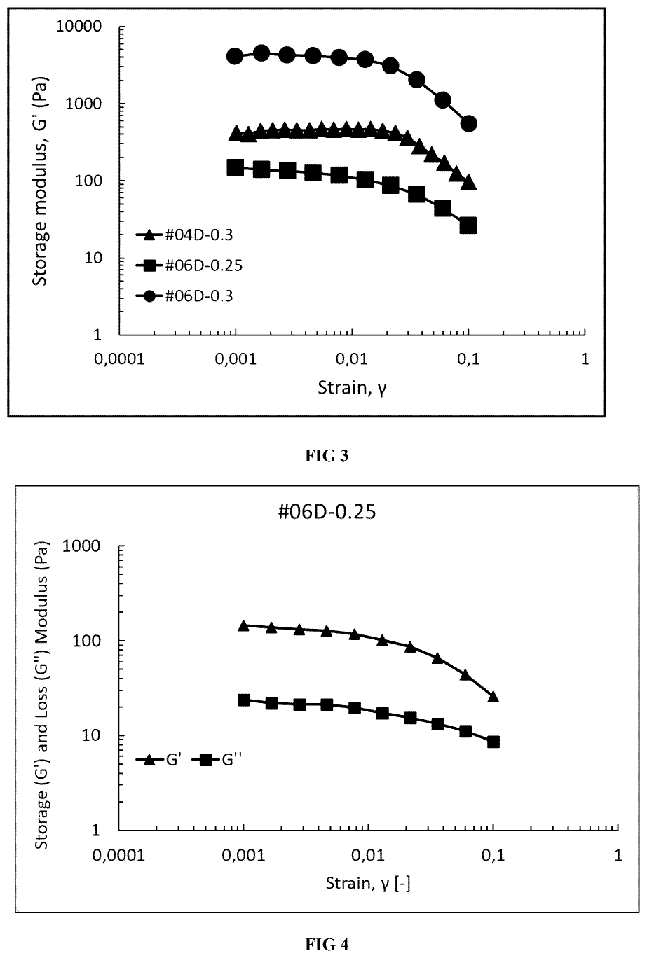

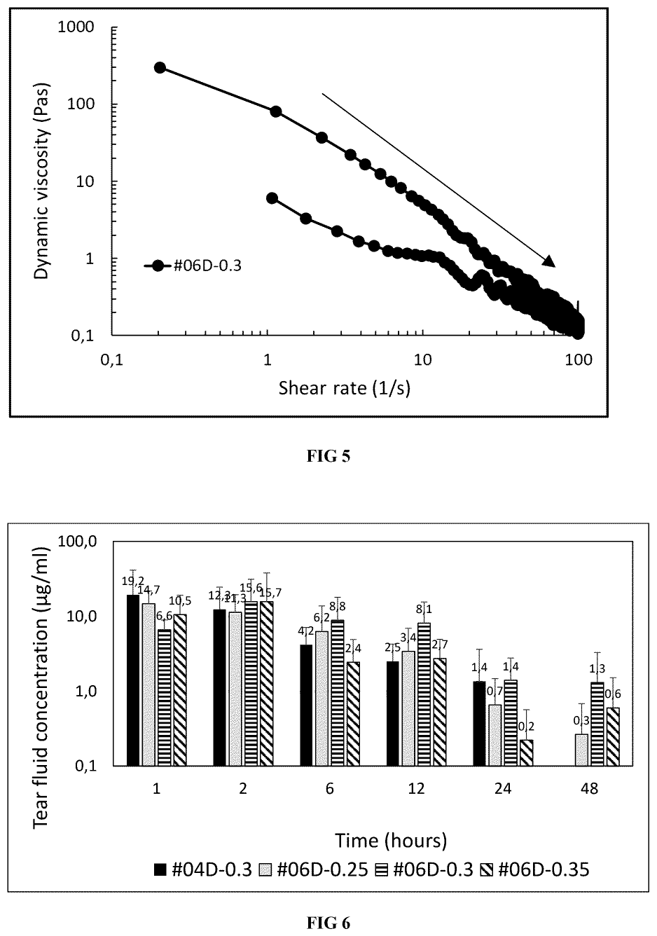

Storage modulus G′ (see FIG. 3) for 3 different formulations (Formulations #04D-0.3, #06D-0.25, #06D-0.3) at room temperature (about 25° C.) were relatively low, at about 100-4000 Pa at strain of 0.001-0.01 and angular frequency of 1 Hz. Depending on the formulation, they were, during injection, either flowing (viscous, but still easily flowing) or directly easily extrudable hydrogels. Also, the loss moduli G″ were low for #04D-0.3, #06D-0.25, #06D-0.3. In all the studied formulations storage modulus was larger than loss modulus in the linear viscoelastic region (at strain of about 0.001-0.01 and angular frequency of 1 Hz), which indicates a non-flowing structure after injection. The storage moduli (about 100-150 Pa) and loss moduli (about 10-20 Pa) for #06-0.25 are shown in FIG. 4. The storage moduli and loss moduli for #04D-0.3 were found to be about 400-460 Pa, and about 15-30 Pa, respectively. The storage moduli and loss moduli for #06D-0.3 were found to be about 3700-4000 Pa and 210-270 Pa, respectively, at strain of 0.001-0.01 and angular frequency of 1 Hz. The low moduli values for the hydrogel composites indicate a loose hydrogel that is easily injectable. This is verified by rotational measurements for dynamic viscosity indicating clear shear-thinning behavior. Dynamic viscosity with a thixotropic behavior (time-dependent change in viscosity due to shear stress) for #06D-0.3 with the strongest hydrogel structure (highest storage modulus) is shown in FIG. 5, and a clear shear-thinning behavior was observed. In addition, some thixotropic behavior was observed, i.e., the dynamic viscosity was a bit lower at same shear rate when going back towards lower shear rates from the maximum of 100 l/s. Corresponding measurements for #04D-0.3 and #06D-0.25 also showed clear shear-thinning, but no or very small thixotropic behavior. Dynamic viscosity was about 1.5 Pas at shear rate of 1.3 l/s and 0.026 Pas at 100 l/s for #06D-0.25, while it was about 6.2 Pas at 1.1 l/s and 0.06 Pas at 100 l/s for #04D-0.3.

In general, the depot gels get weaker with the lower microparticle concentrations in the depot, and particularly below 30% there may sometimes occur phase separation under high shear stress. However, surprisingly in the present formulation all the depots were non-flowing gel structures not only at rest, but also after the administration (which would not necessarily occur at higher shear stresses for weak gels). The above results show that storage (elastic) modulus G′ is surprisingly higher than loss (viscous) modulus G″. This means that the eye drop administration (with relatively low shear stresses) holds the gel structure intact, and the formulation enters the eye in the form of a gel, which does not leak out immediately away. This enables controlled release for a certain time, thereby improved efficacy.

### Example 4

### PK Study of Single Dose Silica Hydrogel Composites in Rabbits and In Vivo Release of Dexamethasone

Three Specific Pathogen Free (SPF) NZW female rabbits (Origin: Lidköpings Kaninfarm, Sweden) were used for the PK study. The study was conducted after seeking approval from the National Laboratory Animal Board of Finland (Care and Use Committee). The Isolation/acclimatisation period was 5 days before the first experiment. The animal room temperature was 21° C.±3° C., relative humidity was at least 55+15%, and the lighting was artificial (12 h light and 12 h dark). The animals were housed in Scanbur no. 8 system, one animal per cage. No randomization was performed.

of a gel, which does not leak out immediately away. This enables controlled release for a certain time, thereby improved efficacy.

### Test Items and Dosing

The hydrogel composite was prepared using silica microparticle of formulation of R3-100 (pH 4.0) with 10% w/w of encapsulated dexamethasone (calculated in relation to the theoretical silica amount), and R400 silica sol (0.3 mg of silica microparticles in 1 ml of silica sol) in Experiment 1. The hydrogel composites prepared using silica microparticle with the formulation of R3-100 (pH 4.0) with 15% w/w of encapsulated dexamethasone (calculated in relation to the theoretical silica amount), and R400 silica sol were used in experiments 2, 3 and 4, containing 0.25 mg, 0.30, and 0.35 of silica microparticles in 1 ml of silica sol, respectively (see Table 2).

TABLE 2
The hydrogel composite formulations for in vivo experiments

| | | | | | Mass of added |
|---|---|---|---|---|---|
| | | | | | microparticles |
| | | | | | in 1 ml |
| | | | | | of silica |
| | | | | Sol for | sol forming a |
| Experiment | | Micro- | API | hydrogel | hydrogel |
| # | Formulation | particles | load* | formation | composite |
| 1 | #04D-0.3 | R3-100, | 10% | R400 | 0.30 g/ml |
| | | pH 4.0 | | | |
| 2 | #06D-0.25 | R3-100, | 15% | R400 | 0.25 g/ml |
| | | pH 4.0 | | | |
| 3 | #06D-0.3 | R3-100, | 15% | R400 | 0.30 g/ml |
| | | pH 4.0 | | | |
| 4 | #06D-0.35 | R3-100, | 15% | R400 | 0.35 g/ml |
| | | pH 4.0 | | | |

*Loading-% (w/w) in relation to the theoretical mass of silica in microparticles

One drop of each hydrogel composite corresponding to 30-40 μl, corresponding to about 16 mg/ml of dexamethasone in Experiment 1, about 26 mg/ml in Experiment 2, about 28 mg/ml in Experiment 3, and about 32 mg/ml in Experiment 4, was placed in the conjunctival sac of both eyes of each rabbit, after gently pulling the lower lid away from the eyeball. The lids were then gently held together for about one second in order to prevent loss of the material. The rabbits were kept in the restrainer for about 1-2 minutes after dosing, after which the rabbits were put back into their own cage where they were able to move freely. Each rabbit was dosed four times with a wash-out period of 1-2 weeks (details in Table 3 below).

TABLE 3

Test Item

| | Exper- | Exper- | Exper- | Exper- |
|---|---|---|---|---|
| Rabbit # | iment 1 | iment 2 | iment 3 | iment 4 |
| 1 | #04D-0.3 | #06D-0.25 | #06D-0.3 | #06D-0.35 |
| 2 | #04D-0.3 | #06D-0.25 | #06D-0.3 | #06D-0.35 |
| 3 | #04D-0.3 | #06D-0.25 | #06D-0.3 | #06D-0.35 |

### Sampling

Tear fluid (2 μl) was collected from rabbit eye at time points 0 (prior to dosing), 1 h, 2 h, 6 h, 12 h, 24 h and 48 h after the dosing. The tear fluid was collected using 2 μl capillary (Microcaps®). The tear fluid was removed from the capillary to the plastic vials using a pipette (pressure technology). 48 μl of 30% acetonitrile solution was added immediately after sample taking into the vials and the vial was sacked in order to mix the tear fluid and acetonitrile. The samples were stored at 4-8° C. until testing.

### In Vivo Release Rate of Dexamethasone

The analysis of dexamethasone in the tear samples was conducted by HPLC measurements to establish an in vivo release profile for dexamethasone. 1260 Infinity II HPLC with a Model G7117C diode array detector from Agilent Technologies was used. The column used was Xbridge C18 2.1×50 mm 2.5 μm from Waters, and the column temperature was 40° C. Water/Trifluoroacetic acid 1000+1 (v/v) was used as mobile phase A, and Acetonitrile/Trifluoroacetic acid 1000+0.9 (v/v) was used as mobile phase B. The gradient run is described in Table 4 below. Flow rate was 0.5 ml/min, wavelength 254 nm, injection volume 20 μl, run time 6.5 min, and the retention time for dexamethasone was 2.9 min. Standards were prepared in 50 mM Tris, pH 7.4. Standards were stored at 4° C.

TABLE 4
Gradient run for dexamethasone analysis

| | Mobile Phase A | Mobile Phase B |
|---|---|---|
| Time (hours) | (%) | (%) |
| 0.00 | 80 | 20 |
| 2.00 | 30 | 70 |
| 2.20 | 1 | 99 |
| 3.20 | 1 | 99 |
| 3.70 | 80 | 20 |
| 6.50 | 80 | 20 |

Tables 5A-5D below illustrates dexamethasone concentrations in tear fluids for all four formulations studied in vivo, showing parallel samples in both eyes of the three rabbits in whom it was tested, as described in Table 3 above. In Tables 5A-5D all concentrations and hourly release are given in μg/ml, SD=Standard deviation.

TABLE 5A
Dexamethasone concentrations in tear fluids for Test Item #04D-0.3

| | Rabbit #1 | | Rabbit #2 | | Rabbit #3 | | | Hourly | |
|---|---|---|---|---|---|---|---|---|---|
| | Left | Right | Left | Right | Left | Right | Average | release | |
| Time | eye | eye | eye | eye | eye | eye | (μg/ml) | (μg/ml) | SD |
| 1 | 13.46 | 5.23 | 6.15 | 26.46 | 3.45 | 60.64 | 19.2 | 19.2 | 22.0 |
| 2 | 2.83 | 1.76 | 22.09 | 16.61 | 29.26 | 1.38 | 12.3 | 12.3 | 12.0 |
| 6 | 0.95 | 1.50 | 2.99 | 4.98 | 5.83 | 8.68 | 4.2 | 1.0 | 2.9 |
| 12 | 1.63 | 0.46 | 1.25 | 2.49 | 4.84 | 4.30 | 2.5 | 0.4 | 1.7 |
| 24 | 0.00 | 0.00 | 0.00 | 2.68 | 5.43 | 0.00 | 1.4 | 0.1 | 2.3 |
| 48 | 0 | 0 | 0 | 0 | 0 | 0 | 0.0 | 0.0 | 0 |

TABLE 5B
Dexamethasone concentrations in tear fluids for Test Item #06D-0.25

| | Rabbit #1 | | Rabbit #2 | | Rabbit #3 | | | Hourly | |
|---|---|---|---|---|---|---|---|---|---|
| | Left | Right | Left | Right | Left | Right | Average | release | |
| Time | eye | eye | eye | eye | eye | eye | (μg/ml) | (μg/ml) | SD |
| 1 | 18.29 | 11.74 | 25.85 | 6.51 | 9.10 | 16.46 | 14.7 | 14.7 | 7.0 |
| 2 | 10.26 | 2.25 | 26.34 | 12.43 | 8.20 | 8.29 | 11.3 | 11.3 | 8.1 |
| 6 | 2.95 | 1.08 | 9.88 | 19.89 | 3.00 | 0.69 | 6.2 | 1.6 | 7.5 |
| 12 | 1.81 | 10.39 | 3.04 | 2.09 | 1.81 | 1.24 | 3.4 | 0.6 | 3.5 |
| 24 | 0.74 | 0.68 | 2.19 | 0.31 | 0.00 | 0.00 | 0.7 | 0.1 | 0.8 |
| 48 | 0.00 | 0.00 | C0.70 | 0.00 | 0.89 | 0.00 | 0.3 | 0.0 | 0.4 |

TABLE 5C
Dexamethasone concentrations in tear fluids for Test Item #06D-0.3

| | Rabbit #1 | | Rabbit #2 | | Rabbit #3 | | | Hourly | |
|---|---|---|---|---|---|---|---|---|---|
| | Left | Right | Left | Right | Left | Right | Average | release | |
| Time | eye | eye | eye | eye | eye | eye | (μg/ml) | (μg/ml) | SD |
| 1 | 8.31 | 3.43 | 7.11 | 4.41 | 9.54 | 6.63 | 6.6 | 6.6 | 2.3 |
| 2 | 5.21 | 5.26 | 4.23 | 10.25 | 26.51 | 42.29 | 15.6 | 15.6 | 15.5 |
| 6 | 3.38 | 17.96 | 3.55 | 2.78 | 22.75 | 2.34 | 8.8 | 2.2 | 9.1 |
| 12 | 8.19 | 21.59 | 1.30 | 3.88 | 10.00 | 3.50 | 8.1 | 1.3 | 7.4 |
| 24 | 0.55 | 0.78 | 0.63 | 0.98 | 4.13 | 1.30 | 1.4 | 0.1 | 1.4 |
| 48 | 0.00 | 1.48 | 1.05 | 0.00 | 5.08 | 0.00 | 1.3 | 0.1 | 2.0 |

TABLE 5D
Dexamethasone concentrations in tear fluids for Test Item #06D-0.35

| | Rabbit #1 | | Rabbit #2 | | Rabbit #3 | | | Hourly | |
|---|---|---|---|---|---|---|---|---|---|
| | Left | Right | Left | Right | Left | Right | Average | release | |
| Time | eye | eye | eye | eye | eye | eye | (μg/ml) | (μg/ml) | SD |
| 1 | 14.53 | 5.00 | 25.83 | 9.03 | 2.75 | 6.08 | 10.5 | 10.5 | 8.5 |
| 2 | 3.73 | 1.20 | 18.58 | 59.05 | 4.25 | 7.15 | 15.7 | 15.7 | 22.1 |
| 6 | 0.58 | 0.30 | 4.63 | 5.55 | 0.00 | 3.55 | 2.4 | 0.6 | 2.4 |
| 12 | 1.03 | 4.05 | 2.48 | 2.60 | 0.00 | 6.18 | 2.7 | 0.5 | 2.2 |
| 24 | 0.70 | 0.00 | 0.00 | 0.00 | 0.00 | 0.63 | 0.2 | 0.0 | 0.3 |
| 48 | 0.00 | 0.00 | 0.83 | 2.33 | 0.00 | 0.43 | 0.6 | 0.0 | 0.9 |

### Clinical Observations

The rabbits were monitored for 48 hours after the dosing of each formulation: #04D-0.3, #06D-0.25, #06D-0.3 and #06D-0.35. The clinical observations made prior to sampling during each study are reported in Tables 6-9.

TABLE 6
Clinical observations made after dosing formulation #04D-0.3

| | Time | Observation |
|---|---|---|
| Test item | (hours) | Study #1 |
| #04D-0.3 | 0 | Rabbit #1 and #2: Eyes open, normal |
| | | Rabbit #3: Eyes closed, stressed. Test item moved from the eye to |
| | | the eyelids within a couple of minutes after dosing. |
| | 1 | Rabbit #1 and #3: Test items appeared on the eyelids. |
| | 2 | Rabbit #2: Test items appeared in the eye (below the eyelid) |
| | 6 | Rabbit #1: Test item in the right eye (below the eyelid) |
| | | Rabbit #2: Test item in the eyes (below the eyelid) |
| | 12 | Nothing reported. |
| | 24 | Rabbit #2: Test item in the right eye (below the eyelid) |
| | 48 | Nothing reported. |

Time point 0 represents the time immediately after dosing.

TABLE 7
Clinical observations made after dosing of formulation #06D-0.25

| | Time | Observation |
|---|---|---|
| Test item | (hours) | Study #2 |
| #06D-0.25 | 0 | Rabbit #1: Irritation for 1-10 minutes, wet eyes, restless. Some test |
| | | item leakage |
| | | Rabbit #2 and #3: Eyes open, normal |
| | 1 | Rabbit #1 and #3: No irritation observed |
| | | Rabbit #2: Test item on the left upper lid |
| | 2 | Rabbit #1 and #2: Test item on the eyes |
| | | Rabbit #3: Test item not visible |
| | 6 | Rabbit #1: Test item on the eyes |
| | 12 | Rabbit #1: Uncertain observation from the test item |
| | 24 | Nothing reported. |
| | 48 | Nothing reported. |

TABLE 8
Clinical observations made after dosing of formulation #06D-0.3

| | Time | Observation |
|---|---|---|
| Test item | (hours) | Study #3 |
| #06D-0.3 | 0 | Rabbit #1: Eyes open, normal. Test item on the eyes |
| | | Rabbit #2: Eyes open, normal. Some test item on top of the left |
| | | eyelid |
| | | Rabbit #3: Eyes open, normal. Test item partly on top of the eye's |
| | | plica semilunaris |
| | 1 | Rabbit #1: Test item on the upper lid of the left eye. Test item partly |
| | | outside of the lower lid of the right eye |
| | | Rabbit #2: Test item partly outside of the lower left eye |
| | | Rabbit #6: Test item partly outside of the lower left eye |
| | 2 | Rabbit #1: Test item on the eyes |
| | | Rabbit #2: Test item on the eyes |
| | | Rabbit #3: Test item partly outside of the lower left eye |
| | 6 | Rabbit #1 and #2: Test item on the eyes |
| | | Rabbit #3: Test item on the left eye |
| | 12 | Rabbit #1: Test item on the eyes |
| | | Rabbit #2: Test item not visible |
| | | Rabbit #3: Test item on the right eye |
| | 24 | Nothing reported. |
| | 48 | Nothing reported. |

TABLE 9
Clinical observations made after dosing of formulation #06D-0.35

| | Time | Observation |
|---|---|---|
| Test item | (hours) | Study #4 |
| #06D-0.35 | 0 | Rabbit #1: Eyes open, normal. Test item on top of the eyelids. Some |
| | | test item leaked out from both eyes |
| | | Rabbit #2: Eyes open, normal. Some test item leaked out from both |
| | | eyes |
| | | Rabbit #3: Eyes open, normal. Test item partly on top of the eye's |
| | | plica semilunaris |
| | 1 | Rabbit #1: Test item outside of the eyelids. Under the upper eye lid |
| | | and in the |
| | | Rabbit #2: Test item on top of the left eye's plica semilunaris |
| | | Rabbit #3: Nothing reported. |
| | 2 | Rabbit #1: Test item on the left eye |
| | | Rabbit #2: Test item on the right eye |
| | | Rabbit #3: Test item on the left eye |
| | 6 | Rabbit #1: Test item on the right eye |
| | 12 | Rabbit #1: Test item on the left eye |
| | 24 | Nothing reported. |
| | 48 | Nothing reported. |

Abnormal clinical signs were not observed in the studies, thereby indicating that the ophthalmic composition does not cause side effects such as irritation and/or reddening of the eyes, and is potentially safe for use over a longer period of time.

### Example 5

### Particle Size Distribution of Silica

Particle size distribution measurements were conducted in 7 samples of the composition comprising dexamethasone using a Sympatec HELOS 2370 laser diffraction instrument. The Particles in Liquid (PIL) method was employed using ethanol as solvent. The particle size distribution of the microparticle formulations are shown in Table 10 below.

TABLE 10
Measured particle size distributions
of the microparticle formulations

| | D10 | | D50 | | D90 | |
|---|---|---|---|---|---|---|
| Formulation | Average | SD | Average | SD | Average | SD |
| code | (μm) | (μm) | (μm) | (μm) | (μm) | (μm) |
| #01 | 2.58 | 0.20 | 13.02 | 2.16 | 38.61 | 12.37 |
| #02 | 1.74 | 0.01 | 5.36 | 0.01 | 11.00 | 0.07 |
| #03 | 2.01 | 0.06 | 7.66 | 0.30 | 23.33 | 3.59 |
| #04 | 1.86 | 0.07 | 7.05 | 0.44 | 28.75 | 18.77 |
| #05 | 1.96 | 0.04 | 6.64 | 0.21 | 19.53 | 2.74 |
| #06 | 1.60 | 0.01 | 5.20 | 0.06 | 14.07 | 1.71 |
| #09 | 0.98 | 0.00 | 2.70 | 0.00 | 7.67 | 0.02 |

### Example 6

### PK Studies for Single Dose Silica Hydrogel Composition in Rabbits and In Vivo Release of Dexamethasone and Comparison to Existing Maxidex® Eye Drop Product

Specific Pathogen Free (SPF) NZW rabbits (Origin: Lidköpings Kaninfarm, Sweden) were used in the PK studies. Two experiments (experiments 1 and 2) with four rabbits each were used to perform PK study of Maxidex® eye drop product, and two experiments (experiments 3 and 4) with three rabbits each were used to perform PK study of the silica hydrogel compositions. The study was approved by the National Laboratory Animal Board of Finland (Care and Use Committee). The Isolation/acclimatisation period was 8 days before the experiment. The animal room temperature was 21° C.+3° C., relative humidity was at least 55+15% and lighting was artificial (12 h light and 12 h dark). The animals were housed in Scanbur no. 8 system, one animal per cage. No randomization was performed.

### Test Items and Dosing

The silica hydrogel composition prepared by combining (i) silica microparticle with the formulation of R3-200 at pH 4.0 (using ethanol as diluent for dilution from R3 to 200) with 7.5% w/w of encapsulated dexamethasone (calculated in relation to the theoretical silica amount) with (ii) R400 silica sol (0.3 g of silica microparticles in 1 ml of silica sol) was used in experiment 3. The silica hydrogel compositions prepared by combining (i) silica microparticle formulation R3-200 at pH 4.0 (using 50 volume-% ethanol in water as diluent for diluting R3 to 200) with 7.5% w/w of encapsulated dexamethasone (calculated in relation to the theoretical silica amount) with (ii) R400 silica sol (0.3 g of silica microparticles in 1 ml of silica sol) was used in experiment 4 (details are included in Table 11 below).

TABLE 11
The silica hydrogel compositions prepared, API = Dexamethasone

| | | | | | Mass of added micro- |
|---|---|---|---|---|---|
| | | | | Sol for | particles in 1 ml of silica |
| Experiment | Hydrogel | Micro- | API | hydrogel | sol forming a hydrogel |
| # | composition | particles | load* | formation | composite |
| 3 | #09D-0.3 | R3-200, | 7.5% | R400 | 0.30 g/ml |
| | | pH 4.0 | | | |
| | | (diluent | | | |
| | | EtOH) | | | |
| 4 | #12D-0.3 | R3-200, | 7.5% | R400 | 0.30 g/ml |
| | | pH 4.0 | | | |
| | | (diluent | | | |
| | | 50 vol-% | | | |
| | | EtOH in | | | |
| | | water) | | | |

*Loading-% (w/w) in relation to the theoretical mass of silica in microparticles

In Experiment 1, a single dose of Maxidex® was administered as one drop of Maxidex®, one in each eye. In Experiment 2, multiple doses of Maxidex® were adminstered-one drop of Maxidex® in each eye every 4 hours, for 24 hours (i.e. a total of 7 times).

One drop of each hydrogel composite corresponding to 30-40 μl, corresponding to about 14 mg/ml of dexamethasone in Experiment 3 and about 17 mg/ml in Experiment 4, was placed in the conjunctival sac of both eyes of each animal, after gently pulling the lower lid away from the eyeball. The lids were then gently held together for about one second in order to prevent loss of the material. The animals were kept in the restrainer for about 1-2 minutes after dosing, after which the animals were put back into their own cage, where they were able to move freely.

### Sampling

Tear fluid (2 μl) was collected from rabbit eye at time points shown in Table 13 below. The tear fluid was collected using 2 μl capillary (Microcaps®). The tear fluid was removed from the capillary to the plastic vials using a pipette (pressure technology). The samples were stored at −20° C. until testing in dry ice.

### In Vivo Release Rate of Dexamethasone

An aliquot of 2 μl of tear fluid sample was mixed with 18 μl of internal standard solution (10 ng/ml cortisol+prednisone in 25% methanol in phosphate buffered saline) before analysis. The analysis of dexamethasone in the tear samples was conducted by UPLC. Acquity UPLC with Xevo TQ-S triple quadrupole MS from Waters was used. The column used was Kinetex Biphenyl 2.1×50 mm 2.7 μm from Phenomenex, and the column temperature was 40° C. 0.025 volume-% acetic acid in water was used as a mobile phase A, and acetonitrile as mobile phase B. The gradient run is described in Table 12. Flow rate was 0.5 ml/min and injection volume was 4 μl.

TABLE 12
Gradient run for dexamethasone analysis

| Time (min) | Mobile Phase A (%) | Mobile Phase B (%) |
|---|---|---|
| 0.00 | 90 | 10 |
| 0.50 | 90 | 10 |
| 1.50 | 50 | 50 |
| 2.00 | 2 | 98 |
| 2.50 | 2 | 98 |
| 3.50 | 90 | 10 |

Tables 13A-13D illustrate dexamethasone concentration in tear fluids for all compositions studied in vivo, showing parallel samples in both eyes. In Tables 13A-13D all concentrations and hourly release are given in μg/ml, SD=Standard deviation.

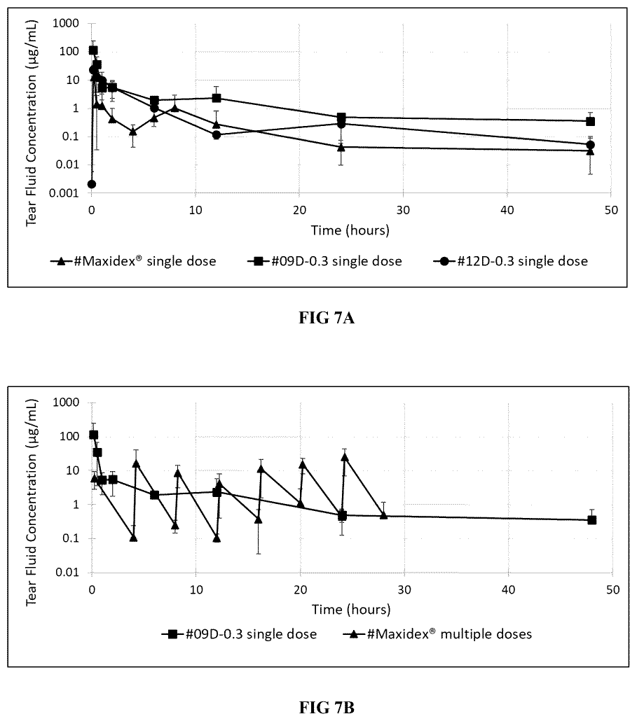

Averages for all results are illustrated in FIGS. 7A and 7B.

TABLE 13A
Dexamethasone concentration in tear
fluids for compositions #09D-0.3

| | Rabbit #1 | | Rabbit #2 | | Rabbit #3 | | | |
|---|---|---|---|---|---|---|---|---|
| Time | Left | Right | Left | Right | Left | Right | | Hourly |
| (h) | eye | eye | eye | eye | eye | eye | Average | release |
| 0.16 | 25.30 | 15.50 | 136.00 | 5.93 | 491.00 | 20.80 | 115.8 | 723.5 |
| 0.5 | 15.10 | 7.65 | 29.20 | 16.80 | 73.50 | 67.60 | 35.0 | 102.9 |
| 1 | 1.24 | 7.01 | 4.24 | 0.98 | 13.40 | 4.41 | 5.2 | 10.4 |
| 2 | 4.35 | 2.20 | 2.51 | 4.17 | 4.28 | 15.10 | 5.4 | 5.4 |
| 6 | 2.77 | 1.43 | 4.10 | 0.41 | 0.66 | 2.22 | 1.9 | 0.5 |
| 12 | 0.31 | 0.85 | 0.30 | 0.11 | 3.97 | 8.36 | 2.3 | 0.4 |
| 24 | 0.54 | 0.12 | 0.18 | 0.68 | 0.55 | 0.82 | 0.5 | 0.0 |
| 48 | 0.00 | 0.24 | 0.28 | 0.07 | 1.48 | 0.03 | 0.3 | 0.0 |

TABLE 13B
Dexamethasone concentration in tear
fluids for compositions #12D-0.3

| | Rabbit #1 | | Rabbit #2 | | Rabbit #3 | | | |
|---|---|---|---|---|---|---|---|---|
| Time | Left | Right | Left | Right | Left | Right | | Hourly |
| (h) | eye | eye | eye | eye | eye | eye | Average | release |
| 0.16 | 24.70 | 22.20 | 26.90 | 17.10 | 35.10 | 14.70 | 23.5 | 146.6 |
| 0.5 | 16.80 | 11.50 | 9.19 | 11.10 | 15.50 | 12.30 | 12.7 | 37.4 |
| 1 | 11.70 | 2.63 | 3.37 | 2.49 | 22.80 | 16.30 | 9.9 | 19.8 |
| 2 | 14.50 | 1.17 | 2.58 | 1.10 | 7.78 | 4.99 | 5.4 | 5.4 |
| 6 | 1.94 | 1.67 | 0.64 | 1.55 | 0.27 | 0.13 | 1.0 | 0.3 |
| 12 | 0.13 | 0.10 | 0.09 | 0.07 | 0.12 | 0.19 | 0.1 | 0.0 |
| 24 | 0.12 | 0.11 | 0.03 | 1.05 | 0.23 | 0.15 | 0.3 | 0.0 |
| 48 | 0.03 | 0.05 | 0.02 | 0.20 | 0.02 | 0.01 | 0.1 | 0.0 |

TABLE 13C
Dexamethasone concentration in tear fluids for Maxidex ® single dose.

| Time | | | | | | Hourly | |
|---|---|---|---|---|---|---|---|
| (h) | Rabbit#1 | Rabbit#2 | Rabbit#3 | Rabbit#4 | Average | Release | SD |
| 0.25 | 41.80 | 5.63 | 1.24 | 2.25 | 12.7 | 50.9 | 19.5 |
| 0.5 | 3.37 | 0.98 | 0.18 | 1.09 | 1.4 | 5.6 | 1.4 |
| 1 | 4.17 | 0.27 | 0.27 | 0.22 | 1.2 | 2.5 | 2.0 |
| 2 | 1.28 | 0.04 | 0.07 | 0.28 | 0.4 | 0.4 | 0.6 |
| 4 | 0.29 | 0.11 | 0.03 | 0.18 | 0.2 | 0.1 | 0.1 |
| 6 | 1.34 | 0.36 | 0.04 | 0.07 | 0.5 | 0.2 | 0.6 |
| 8 | 3.87 | 0.11 | 0.02 | 0.02 | 1.0 | 0.5 | 1.9 |
| 12 | 1.03 | 0.02 | 0.02 | 0.01 | 0.3 | 0.1 | 0.5 |
| 24 | 0.05 | 0.04 | 0.00 | 0.08 | 0.0 | 0.0 | 0.0 |
| 48 | 0.11 | 0.00 | 0.01 | 0.01 | 0.0 | 0.0 | 0.1 |

TABLE 13D
Dexamethasone concentration in tear fluids for Maxidex ® multiple dose.

| Time | | | | | | Hourly | |
|---|---|---|---|---|---|---|---|
| (h) | Rabbit#1 | Rabbit#2 | Rabbit#3 | Rabbit#4 | Average | Release | SD |
| 0.25 | 9.59 | 7.28 | 1.92 | 5.24 | 6.0 | 24.0 | 3.3 |
| 4 | 0.30 | 0.06 | 0.02 | 0.05 | 0.1 | 0.0 | 0.1 |
| 4.25 | 4.44 | 8.07 | 1.57 | 51.20 | 16.3 | 65.3 | 23.4 |
| 8 | 0.21 | 0.25 | 0.14 | 0.38 | 0.2 | 0.1 | 0.1 |
| 8.25 | 3.63 | 5.73 | 8.64 | 16.10 | 8.5 | 34.1 | 5.5 |
| 12 | 0.08 | 0.09 | 0.13 | 0.13 | 0.1 | 0.0 | 0.0 |
| 12.25 | 2.36 | 9.61 | 2.64 | 1.75 | 4.1 | 16.4 | 3.7 |
| 16 | 0.51 | 0.78 | 0.09 | 0.10 | 0.4 | 0.1 | 0.3 |
| 16.25 | 3.38 | 3.36 | 14.80 | 23.20 | 11.2 | 44.7 | 9.7 |
| 20 | 0.18 | 0.22 | 0.25 | 3.75 | 1.1 | 0.3 | 1.8 |
| 20.25 | 7.50 | 25.20 | 15.00 | 13.10 | 15.2 | 60.8 | 7.4 |
| 24 | 0.45 | 0.86 | 0.15 | 0.27 | 0.4 | 0.1 | 0.3 |
| 24.25 | 15.60 | 9.67 | 50.60 | 24.00 | 25.0 | 99.9 | 18.1 |
| 28 | 0.10 | 1.50 | 0.08 | 0.30 | 0.5 | 0.1 | 0.7 |

### Clinical Observations

The rabbits were monitored for 48 hours after the dosing of silica hydrogel compositions #09D-0.3 and #12D-0.3. The clinical observations made prior to sampling during each study are reported in tables 14 and 15.

TABLE 14
Clinical observations made after dosing formulation #09D-0.3

| | Time | Observation |
|---|---|---|
| Test item | (hours) | Experiment #3 |
| #09D-0.3 | Immediately | Rabbit #3: Washed eyes after dosing. It seemed that part of the |
| | after dosing | drop came out immediately after dosing. |
| | | Rabbit #4: Washed eyes after dosing. |
| | 0.17 | Rabbit #1 and #3: Test item on the eye |
| | 0.5 | Rabbit #1, #3, #4: Nothing reported |
| | 1 | Rabbit #1, #3, #4: Nothing reported |
| | 2 | Rabbit #1 and #3: Test item on the eye |
| | | Rabbit #4: Test item not visible |
| | 6 | Rabbit #1, #3 and #4: Nothing reported |
| | 12 | Rabbit #1 and #3: Test item not visible |
| | | Rabbit #4: Nothing reported |

TABLE 15
Clinical observations made after dosing formulation #12D-0.3

| | Time | Observation |
|---|---|---|
| Test item | (hours) | Study #4 |
| #12D-0.3 | Immediately | Rabbit #1, #3 and #4: Washed eyes after dosing In the third |
| | after dosing | eyelid, slight redness |
| | 0.17 | In the third eyelid, slight redness |
| | 0.5 | In the third eyelid, slight redness |
| | 1 | In the third eyelid, slight redness |
| | 2 | Rabbit #1 and #3: Test item on the eyes. In the third eyelid, |
| | | slight redness |
| | | Rabbit #4: Test item on the left eye. In the third eyelid, slight |
| | | redness |
| | 6 | Rabbit #1: Test item on the left eye. In the third eyelid, slight |
| | | redness |
| | | Rabbit #3: Test item on the eyes. In the third eyelid, slight |
| | | redness |
| | | Rabbit #4: Test item on the right eye. In the third eyelid, slight |
| | | redness |
| | 12 | Rabbit #1, #3 and #4: Nothing reported |

No abnormal clinical signs were observed in the studies. This indicates that the ophthalmic compositions are safe for use and do not cause irritation or other side effects.

### Example 7

Accuracy of the dosing units was tested with two silica hydrogel compositions (#09D-0.3 and #12D-0.3). Testing was performed by dropping a single drop from a single dose unit (each single dose unit was used only once) into a 160 ml container. Weight of the sample was recorded, and the sample was completely dissolved in 150 ml 50 mM glycine buffer (pH 9.4 at 37° C.) at 37° C. over 3 days. API content of the sample solution was measured with HPLC.

TABLE 16
Dosing accuracy of the single dose unit
system, mass and concentrations

| | API | Sample | Sample | | Sample | Sample API | |
|---|---|---|---|---|---|---|---|
| Formulation | load¹ | mass | mass (mg) | | API load | load (mg/ml) | |
| Code | (mg/ml) | (mg) | AVG² | SD | (mg/ml) | AVG³ | SD |
| #09D-0.3 | 14 | 36.0 | 38.4 | 2.1 | 11.2 | 14.8 | 3.1 |
| | | 39.7 | | | 16.7 | | |
| | | 39.5 | | | 16.5 | | |
| #12D-0.3 | 17 | 37.8 | 36.6 | 1.1 | 17.6 | 17.0 | 2.4 |
| | | 35.7 | | | 19.0 | | |
| | | 36.4 | | | 14.4 | | |

¹Calculated concentration of API in the final depot formulation
²Average of three replicate samples
³Measured API concentration of sample, average of three replicate samples (average = AVG, and standard deviation = SD)

Both formulations were dosed quite accurately from the single dose units, showing only a minor deviation in the dexamethasone concentration per dose.

### Example 8

### Storage Stability Testing for Dexamethasone Hydrogel Silica Eye Drop Composition

Storage stability of the drug substance and drug product (composition) were studied for a representative formulation #09. Silica-Dexamethasone microparticles were mixed with R400 silica sol in 0.3 weight-volume ratios (w/v). This suspension was then transferred into the single dose units (SDU) through a 20 G needle. After the filling, the SDUs were allowed to gel for 2-3 days. Samples were packed in aluminium pouches and gamma irradiated with an irradiation dose of dose 25.09-26.06 kGy) before starting stability study.

One of the objectives of the study was to evaluate how the quality of drug substance and drug product varies over time under the influence of various storage conditions. Chemical and physical stability of the formulation is assessed in two conditions of storage: 2-8° C. and 25° C.+2° C./60%+5% Relative Humidity (RH) over the duration of three months (with monthly testing frequency). The microparticles and formulation #09 were assessed for stability on various parameters. Reverse-Phase HPLC (RP-HPLC) method and parameters used for analyse of Dexamethasone and related substances is given in Table 17 below. The results of the storage stability study are provided in Tables 18-21 and FIGS. 8A-8B.

TABLE 17
RP-HPLC method for Dexamethasone and Related Substances

| System | 1260 Infinity HPLC Agilent Technologies | | |
|---|---|---|---|
| Detector | Model G7117C Dicode Array Detector, Agilent | | |
| | Technologies | | |
| Column | Waters X Bridge BEH C18, 4/6 × 150 mm, 2.5 μm | | |
| Column temperature (°C) | 45 | | |
| Mobile Phase A | 25% Acetonitrile in H₂O | | |
| Mobile Phase B | 100% Acetonitrile | | |
| | Time (min) | Mobile Phase A (%) | Mobile Phase B (%) |
| Gradient | 0.00 | 100 | 0 |
| | 18.00 | 100 | 0 |
| | 48.00 | 0 | 100 |
| | 48.1 | 100 | 0 |
| | 55 | 100 | 0 |

| | |
|---|---|
| Flow rate (ml/min) | 1 |
| Wavelength (nm) | 254 |
| Injection volume (μl) | 30 |
| Runtime (min) | 55 |

TABLE 18
Dexamethasone Microparticles stability data (R3-200)

| | Sample timepoint | | | | | | |
|---|---|---|---|---|---|---|---|
| | | 1 M | | 2 M | | 3 M | |
| | | | 25° ± 2 °C/ | | 25° ± 2 °C/ | | 25° ± 2 °C/ |
| | 0 M | 2-8 °C | 60% RH ± 5% | 2-8 °C | 60% RH ± 5% | 2-8 °C | 60% RH ± 5% |
| Appearance | White | White | White | White | White | White | White |
| | suspension | powder | powder | powder | powder | powder | powder |
| pH | 6.0 | 5.8 | 5.7 | 5.6 | 5.7 | 5.7 | 5.8 |
| PSD | | | | | | | |
| D10 (μm) | 0.98 | 0.91 | 0.91 | 0.87 | 0.96 | 0.89 | 0.94 |
| D50 (μm) | 2.70 | 2.57 | 2.42 | 2.41 | 2.53 | 2.32 | 2.45 |
| D90 (μm) | 7.67 | 7.66 | 5.69 | 8.23 | 5.92 | 5.36 | 5.62 |
| Area Purity (%) | 99.73 | 99.73 | 99.16 | 99.75 | 98.71 | 99.75 | 97.81 |
| Related | 0.70 | 0.27 | 0.84 | 0.25 | 1.29 | 0.25 | 2.19 |
| substances (%) | | | | | | | |
| Mass balance (%) | N/A | 99 | 101 | 91 | 93 | 97 | 96 |

TABLE 19
Related substances analysis during storage stability of microparticulates (R3-200)

| | Relative Retention Time- | | | | | | |
|---|---|---|---|---|---|---|---|
| | Area Purity (%) in Microparticles | | | | | | |
| | | 1 M | | 2 M | | 3 M | |
| | | 2-8 | 25° ± 2 °C/ | 2-8 | 25° ± 2 °C/ | 2-8 | 25° ± 2 °C/ |
| | 0 M | °C | 60% RH ± 5% | °C | 60% RH ± 5% | °C | 60% RH ± 5% |
| 0.31 | — | — | — | — | — | — | — |
| 0.45 | — | — | — | — | — | — | 0.05 |
| 0.69/0.74 | — | — | — | — | — | — | — |
| 0.87 | — | — | — | — | — | — | — |
| 0.89/0.90⁽² | 0.16 | 0.17 | 0.21 | 0.14 | 0.18 | 0.15 | 0.21 |
| 1.38 | 0.06 | 0.05 | 0.05 | — | — | — | — |
| 1.44⁽⁴ | — | — | 0.05 | — | — | — | 0.11 |
| 1.56/1.57 | 0.05 | 0.05 | 0.05 | 0.05 | 0.05 | 0.05 | 0.05 |
| 1.59 | — | — | — | — | — | — | 0.05 |
| 1.60 | — | — | — | — | — | — | — |
| 1.71 | — | — | — | — | — | — | |
| 1.79/1.80 | — | — | 0.43 | — | 0.89 | 0.05 | 1.53 |
| 1.81 | — | — | 0.06 | — | 0.11 | — | 0.18 |
| 1.92 | — | — | — | — | — | — | — |
| 2.12 | — | — | — | — | — | — | — |
| 2.20 | — | — | — | — | — | — | — |
| Total impurity | 0.27 | 0.27 | 0.84 | 0.25 | 1.29 | 0.25 | 2.19 |
| API | 99.73 | 99.73 | 99.16 | 99.75 | 98.71 | 99.75 | 97.81 |

—: not detected or area percentage is under 0.05%.
1): Purity-% is calculated as a relation of main peak area to total area of main peak and area of peaks of related substances (peaks which area percent are under 0.05% are not included in the calculations).
⁽²Co-eluting impurity peaks in 25 °C and 40 °C samples, which cannot be separated with current method.
3): RRT varies a little bit in different runs due to the shift of API retention time.
⁽⁴matrix interference in some samples.

TABLE 20
Storage stability data of Formulation #09

| | Sample timepoint | | | | | | |
|---|---|---|---|---|---|---|---|
| | | 1 M | | 2 M | | 3 M | |
| | | | 25° ± 2 °C/ | | 25° ± 2 °C/ | | 25° ± 2 °C/ |
| | 0 M | 2-8 °C | 60% RH ± 5% | 2-8 °C | 60% RH ± 5% | 2-8 °C | 60% RH ± 5% |
| Appearance | White | White | White | White | White | White | White |
| | suspension | suspension | suspension | suspension | suspension | suspension | suspension |
| pH | 6.0 | 6.0 | 6.0 | 6.0 | 5.7 | 5.9 | 5.8 |
| Area Purity (%) | 99.73 | 99.25 | 99.27 | 99.19 | 99.19 | 99.16 | 99.22 |
| Related substances (%) | 0.27 | 0.75 | 0.73 | 0.81 | 0.81 | 0.84 | 0.78 |
| Mass balance (%) | 100 | 100 | 96 | 102 | 92 | 98 | 101 |

TABLE 21
Related substances analysis during storage stability of Formulation #09

| | Relative Retention Time- | | | | | | |
|---|---|---|---|---|---|---|---|
| | Area Purity⁽¹ (%) in depot (Eye drop) | | | | | | |
| | | 1 M | | 2 M | | 3 M | |
| | | 2-8 | 25° ± 2 °C/ | 2-8 | 25° ± 2 °C/ | 2-8 | 25° ± 2 °C/ |
| | 0 M | °C | 60% RH ± 5% | °C | 60% RH ± 5% | °C | 60% RH ± 5% |
| 0.49 | — | 0.06 | — | 0.06 | 0.05 | 0.06 | — |
| 0.89⁽² | 0.24 | 0.20 | 0.22 | 0.25 | 0.25 | 0.25 | 0.26 |
| 1.39⁽³ | 0.05 | 0.05 | 0.05 | 0.05 | 0.05 | 0.06 | 0.06 |
| 1.57⁽³ | 0.06 | 0.07 | 0.06 | 0.07 | 0.06 | 0.07 | 0.06 |
| 1.59⁽³ | 0.29 | 0.29 | 0.30 | 0.30 | 0.31 | 0.30 | 0.31 |
| 1.62⁽³ | 0.06 | 0.09 | 0.10 | 0.09 | 0.09 | 0.10 | 0.09 |
| Total impurity | 0.70 | 0.75 | 0.73 | 0.81 | 0.81 | 0.84 | 0.78 |
| API | 99.30 | 99.25 | 99.27 | 99.19 | 99.19 | 99.16 | 99.22 |

—: detected but area percent is under 0.05%.
⁽¹Purity-% is calculated as a relation of main peak area to total area of main peak and area of peaks of related substances (peaks which area percent are under 0.05% are not included in the calculations).
⁽²co-eluting impurity peaks in samples, which cannot be separated with current method.
⁽³RRT varies a little bit in different runs due to the shift of API retention time.

Results: The visual appearance of the microparticles and formulation #09, the API content, silica content, level of non-encapsulated API, pH, and particle size distribution remained unchanged in both storage conditions upto 3 months (Table 17 and 19). No significant API degradation is observed in the microparticles or formulation #09 stored at 2-8° C. upto 3 months, although a slight degradation was observed at 25° C./60% RH condition with time (Table 18 and 20). Cumulative in vitro in sink silica matrix dissolution rate of the depot formulation remains stable at 2-8° C. and 25° C./60% RH at least upto 2 months of storage. Overall, it was surprisingly found that despite having a low silica microparticle wt-%, the formulations remained stable during storage conditions.

### Example 9

### Sustained Release Silica Hydrogel Ophthalmic Composition of Oflaxaxin

Two silica hydrogel compositions of ofloxacin were prepared by combining (i) silica microparticle with the formulation of R3-200 at pH 4.0 (using 80% ethanol as diluent for dilution from R3 to 200) with 5% w/w of encapsulated ofloxacin (calculated in relation to the theoretical silica amount) with (ii) R400 silica sol (0.3 g of silica microparticles in 1 ml of silica sol (Ofloxacin #04 D-0.3) and 0.4 g of silica microparticle in 1 ml of silica sol (Ofloxacin #04 D-0.4)). This suspension was then transferred into the single dose units (SDU) through a 20 G needle. After the filling, the SDUs are allowed to gel for 1 day.

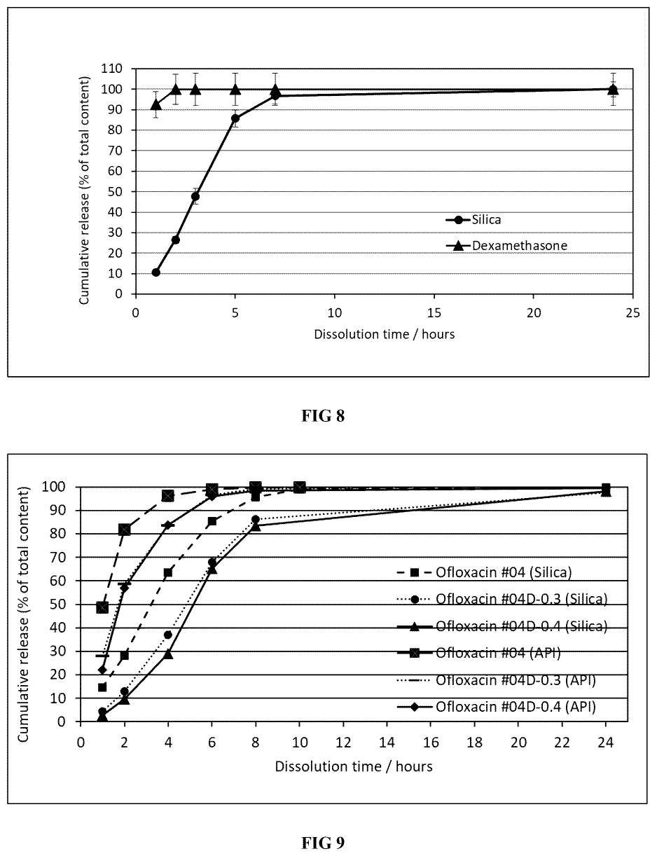

Results: The release profile from these microparticle formulations of ofloxacin is presented in FIG. 9. The slowing effect of the hydrogel component was seen in the both depot formulations, and ofloxacin was released in 8-10 hours in both depot formulations.

It will be appreciated that the compositions and methods provided herein can be incorporated in the form of a variety of embodiments, only a few of which are disclosed herein. It will be apparent to the persons of skill in the art that other embodiments exist, and that the described embodiments are illustrative and should not be construed as restrictive.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting.

Unless the context indicates otherwise, it is specifically intended that the various features of the disclosure described herein can be used in any combination.

Moreover, the present disclosure also contemplates that in some embodiments of the disclosure, any feature or combination of features set forth herein can be excluded or omitted.

All publications, patent applications, patents, and other references mentioned herein are incorporated by reference herein in their entirety for all purposes.

As used herein, “a,” “an,” or “the” can mean one or more than one.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this disclosure, dose, time, temperature, pH and the like, is meant to encompass variations of +20% of the specified amount.

1. A hydrogel silica composition comprising:
a) silica microparticles comprising an active pharmaceutical ingredient, and having a maximum diameter in a range of about 0.5 μm to about 40 μm, and
b) a silica sol comprising solid nanoparticles of <50 nm;
wherein
i) said silica sol has a solid content of <1% by weight,
ii) said hydrogel silica composition comprises up to 30% by weight of the composition of said silica microparticles, and
iii) said hydrogel silica composition is for topical ophthalmic administration.

2. The hydrogel silica composition of claim 1, wherein the active pharmaceutical ingredient is selected from an anti-inflammatory agent, corticosteroid, non-steroidal anti-inflammatory compound, immunosuppressants, antibiotics, lymphocyte function-associated antigen-1 (LFA-1) antagonists, or a recombinant-human nerve growth factor.

3. The hydrogel silica composition of claim 2, wherein the corticosteroid is selected from prednisolone, dexamethasone, fluocinolone, fluorometholone, medrysone or rimexolone, or their pharmaceutically acceptable salts, preferably the corticosteroid is dexamethasone or its pharmaceutically acceptable salt.

4. The hydrogel silica composition of claim 2, wherein the non-steroidal anti-inflammatory compound is selected from ketorolac, flurbiprofen, bromfenac, diclofenac, nepafenac or their pharmaceutically acceptable salt, preferably the non-steroidal anti-inflammatory compound is nepafenac or its pharmaceutically acceptable salt.

5. The hydrogel silica composition of claim 2, wherein the immunosuppressant is cyclosporin or voculosporin, or a pharmaceutically acceptable salt thereof, preferably the immunosuppressant is cyclosporin or its pharmaceutically acceptable salt.

6. The hydrogel silica composition of claim 2, wherein the antibiotic is ofloxacin or its pharmaceutically acceptable salt.

7. The hydrogel silica composition of claim 2, wherein the recombinant-human nerve growth factor is cenegermin.

8. The hydrogel silica composition of any one of the preceding claims 1-7, wherein the silica microparticles have a maximum diameter in a range of about 1 to about 40 μm, preferably 1-30 μm, more preferably 1-20 μm.

9. The hydrogel silica composition of any one of the preceding claims 1-8, wherein the silica microparticles have a maximum diameter in a range of about 0.9 to about 40 μm, preferably 0.9-30 μm, more preferably 0.9-20 μm, and even more preferably 0.9-10 μm.

10. The hydrogel silica composition of any one of the preceding claims 1-8, wherein the silica microparticles have an average diameter D10 in a range of 0.9-10 μm and/or average diameter D50 in a range of 0.5-15 μm and/or average diameter D90 in a range of 5-40 μm, preferably 5-20 μm.

11. The hydrogel silica composition of any one of the preceding claims 1-10, wherein the silica microparticles comprise up to 30 wt-%, preferably in a range of 0.1-30 wt-%, more preferably 0.5-20 wt-%, even more preferably 1.5-15 wt-%, and most preferably 3-7.5 wt-% of the active pharmaceutical ingredient.

12. A hydrogel silica composition comprising:
a) silica microparticles containing about ≤15 wt-%, preferably 10 wt-%, more preferably ≤7.5 wt-% of dexamethasone or its pharmaceutically acceptable salt, wherein the silica microparticles have an average diameter D10 in a range of 0.9-10 μm and/or average diameter D50 in a range of 0.5-15 μm and/or average diameter D90 in a range of 5-40 μm, preferably 5-20 μm; and
b) a silica sol comprising solid nanoparticles of <50 nm, wherein the silica sol has a solid content of <1% by weight, and
wherein the hydrogel silica composition comprises up to 30% by weight of the composition of said silica microparticles.

13. The hydrogel silica composition of any one of the preceding claims 1-12, wherein the composition is non-flowing upon administration to an eye.

14. The hydrogel silica composition of any one of the preceding claims 1-13, wherein storage (elastic) modulus G′ of the composition is higher than loss (viscous) modulus G″.

15. The hydrogel silica composition of any one of the preceding claims 1-14, wherein the composition is a sustained release composition.

16. The hydrogel silica composition of any one of the preceding claims 1-15, wherein the composition remains stable for a period of at least 1 month, preferably at least 2 months, and more preferably at least 3 months upon storage at 2-8° C.

17. The hydrogel silica composition of any one of the preceding claims 1-16, wherein the composition is provided in the form of topical eye drops.

18. The hydrogel silica composition of any one of the preceding claims 1-16, wherein the composition is provided in a single dose container.

19. The hydrogel silica composition of any one of the preceding claims 1-18 for use in treatment of an eye disorder or an eye disease by topical administration.

20. The hydrogel silica composition for use of claim 19, wherein the eye disorder or eye disease is a corneal injury from chemical, radiation or thermal burns or following penetration by foreign bodies.

21. The hydrogel silica composition for use of claim 19, wherein the eye disorder or eye disease is ocular inflammation.

22. The hydrogel silica composition for use of claim 21, wherein the ocular inflammation is selected from anterior uveitis, iritis, cyclitis, allergic or vernal conjunctivitis, herpes zoster keratitis, superficial punctate keratitis or non-specific superficial keratitis, or post-operative ocular inflammation.

23. The hydrogel silica composition for use of any one of the preceding claims 19-22, wherein the composition provides sustained release of the active pharmaceutical ingredient contained therein.

24. The hydrogel silica composition for use of any one of the preceding claims 19-23, wherein the active pharmaceutical ingredient is dexamethasone or its pharmaceutically acceptable salt.

25. The hydrogel silica composition of claim 12 for use in treatment of post-operative ocular inflammation.

26. The hydrogel silica composition for use of any one of the claims 19-25, wherein the composition is topically administered once-a-day to a patient in need thereof.

27. A method for preparing a hydrogel silica composition wherein silica microparticles, comprising an active pharmaceutical ingredient, and having a maximum diameter in a range of about 0.5 μm to about 40 μm, are mixed with a silica sol such that:
i) said silica sol has a solid content of ≤1% by weight, and
ii) said hydrogel silica composition comprises up to 30% by weight of the composition of said silica microparticles.

28. The method of claim 27, wherein the silica microparticles are obtained by a process of spray drying the silica with the active pharmaceutical ingredient.

29. The method of claim 27 or 28, wherein the active pharmaceutical ingredient is selected from an anti-inflammatory agent, corticosteroid, non-steroidal anti-inflammatory compound, immunosuppressants, antibiotics, lymphocyte function-associated antigen-1 (LFA-1) antagonists, or a recombinant-human nerve growth factor.

30. The method according to claim 29, wherein the corticosteroid is selected from prednisolone, dexamethasone, fluocinolone, fluorometholone, medrysone or rimexolone, or their pharmaceutically acceptable salts, preferably the corticosteroid is dexamethasone or its pharmaceutically acceptable salt.

31. The method composition according to claim 29, wherein the non-steroidal anti-inflammatory compound is selected from ketorolac, flurbiprofen, bromfenac, diclofenac, nepafenac or their pharmaceutically acceptable salt, preferably the non-steroidal anti-inflammatory compound is nepafenac or its pharmaceutically acceptable salt.

32. The method according to claim 29, wherein the immunosuppressant is cyclosporin or voculosporin, or a pharmaceutically acceptable salt thereof, preferably the immunosuppressant is cyclosporin or its pharmaceutically acceptable salt.

33. The method according to claim 29, wherein the antibiotic is ofloxacin or its pharmaceutically acceptable salt.

34. The method according to claim 29, wherein the recombinant-human nerve growth factor is cenegermin.

35. A hydrogel silica composition obtainable by the method of any one of the preceding claims 27-34.

36. A method of treatment of an eye disorder or an eye disease in a patient in need thereof, the method comprising topically administering to the patient a hydrogel silica composition of any one of the preceding claims 1-18.

37. A method of treatment of ocular inflammation comprising topically administering the hydrogel silica composition of claim 12.

38. The of claim 37, wherein the ocular inflammation is selected from anterior uveitis, iritis, cyclitis, allergic or vernal conjunctivitis, herpes zoster keratitis, superficial punctate keratitis or non-specific superficial keratitis or post-operative ocular inflammation.

39. Use of a hydrogel silica composition of any one of the preceding claims 1-18 for treatment of an eye disorder or an eye disease.

40. Use of a hydrogel silica composition of claim 12 for treatment of ocular inflammation.

41. Use of claim 40, wherein the ocular inflammation is selected from anterior uveitis, iritis, cyclitis, allergic or vernal conjunctivitis, herpes zoster keratitis, superficial punctate keratitis or non-specific superficial keratitis or post-operative ocular inflammation.
